(19) United States
(12) Patent Application Publication
(10) Pub. No.: US 2022/0062183 A1
(43) Pub. Date: Mar. 3, 2022

(54) PROCESS FOR THE MANUFACTURE OF A TABLET OF RIFAXIMIN AND TABLET OF RIFAXIMIN

(71) Applicant: Friulchem S.p.A., Vivaro PN (IT)

(72) Inventors: Disma Giovanni MAZZOLA, Milano MI (IT); Teresa DI PETRILLO, Milano MI (IT)

(21) Appl. No.: 17/414,487

(22) PCT Filed: Dec. 19, 2018
(86) PCT No.: PCT/IB2018/060357

§ 371 Date: Jun. 16, 2021

(51) Int. Cl. A61K 9/20; A61K 31/437; A61K 9/28

(52) U.S. Cl. CPC A61K 9/2095; A61K 31/437; A61K 9/28; A61K 9/2013; A61K 9/2059; A61K 9/2009; A61K 9/2054

(57) ABSTRACT

The present invention refers to a process for the manufacturing of a tablet of rifaximin starting from a pseudo-crystalline form of rifaximin, as well a tablet of rifaximin obtainable by such process.

### FIELD OF TECHNOLOGY

This invention pertains to the field of pharmaceuticals, in particular to antibiotics for the gastrointestinal tract and to processes for manufacturing tablets.

### STATE OF THE ART

Rifaximin is an antibiotic typically used to treat bacterial infections of the gastrointestinal tract. Its indication includes traveler's diarrhea, irritable bowel syndrome, and hepatic encephalopathy.

Many polymorphic forms of rifaximin have been discovered. International patent application No. PCT/EP2012/059404, in the name of the same applicant of the present invention, discloses a pseudo-crystalline form and the process for its preparation. Such pseudo-crystalline form has many favorable features, such as increased stability in the presence of water and low amount of impurities contained within it.

Rifaximin has to be administered via oral route if it has treat the gastrointestinal tract. Rifaximin can exert its antibiotic effect in the gastrointestinal tract only if it is not absorbed, or if it is absorbed in low amounts, in the gastrointestinal tract during oral administration. An oral dosage form comprising the above-mentioned pseudo-crystalline form having suitable characteristics has been found difficult to formulate by conventional processes.

There is therefore the need to find effective processes for the manufacture of a suitable dosage form of rifaximin starting from its advantageous pseudo-crystalline form.

### DESCRIPTION OF THE INVENTION

Object of the present invention is to provide a process for the manufacture of a suitable oral dosage form starting from pseudo-crystalline rifaximin, i.e. an oral dosage form that has suitable characteristics to be supplied, stored, administered to a patient, and that exerts an antibiotic effect in the gastrointestinal tract.

Also object of the present invention is to provide an oral dosage form starting from pseudo-crystalline rifaximin that has the suitable characteristics mentioned above.

These objects as well as others are achieved by the subject-matter of the present invention, namely a process for the manufacturing of a tablet of rifaximin comprising the following steps:
a) providing a mixture comprising pseudo-crystalline rifaximin and suitable pharmaceutical excipients;
b) granulating the mixture of step a) by wet granulation in a high shear mixer granulator, whereby granules are obtained;
c) drying the granules obtained in step b), whereby dry granules are obtained; and
d) tableting the dry granules of step c), whereby the tablet of rifaximin are obtained;

wherein the granulating step (step b)) is carried out with an impeller speed of 1000 rpm or more, preferably the impeller speed is comprised in the range of 1000 to 2000 rpm, more preferably is 1500 rpm.

The granulating step, i.e. step b) of the process of the invention, is preferably carried out for a time longer than 10 minutes, more preferably for a time comprised in the range of 10 to 20 minutes and even more preferably of 15 minutes.

As referred herein, “pseudo-crystalline rifaximin” refers to the API rifaximin having an XRPD pattern with main peaks at about 5.9°, 7.3°, 7.9° and 8.4° 2θ, preferably at about 5.9°, 7.3°, 7.8° 7.9°, 8.0° 8.4° and 8.6° 2θ, and more preferably at about 5.2°, 5.9°, 6.3°, 6.9°, 7.3°, 7.6° 7.8° 7.9°, 8.0°, 8.4°, 8.6° and 9.0°2θ. A XRPD pattern of the API pseudo-crystalline rifaximin is shown in FIG. 1. A complete list of peaks of the XRPD pattern of the API pseudo-crystalline rifaximin is shown in FIG. 2.

The method for producing the pseudo-crystalline form of rifaximin is disclosed on International patent application No. PCT/EP2012/059404, to which reference is made.

As stated above, the pseudo-crystalline rifaximin has numerous advantages, such as increased stability in the presence of water and low amount of impurities contained within it. Therefore, providing a dosage form of rifaximin starting from the pseudo-crystalline rifaximin is likewise advantageous. As stated above, rifaximin is mostly used as an antibiotic for the gastrointestinal tract, as it can be formulated into oral dosage forms that provide low gastrointestinal absorption of rifaximin and therefore high amount of rifaximin in the gastrointestinal tract that will exert its antibiotic effects. The amount of rifaximin absorbed when an oral dosage form of rifaximin is administered depends on the polymorphic form of rifaximin and on the manufacturing process of the oral dosage form.

It has been found that conventional tableting processes to obtain a tablet of rifaximin starting from the pseudo-crystalline rifaximin provide tablets with inappropriate physical and/or pharmacokinetic (e.g. absorption) properties. For example, conventional tableting processes comprising direct compression or dry granulation resulted not feasible, as they provided tablets with unsuitable hardness. Accordingly, conventional tableting processes comprising wet granulation without using the above-mentioned parameters of the process of the invention provided rifaximin tablets lacking suitable physical characteristics to be administered to patients, in particular lacking suitable hardness and friability.

The above-mentioned problems are solved by carrying out the process of the invention. Indeed, it has been surprisingly found out that the parameters above provide tablets of rifaximin starting from its advantageous pseudo-crystalline form having suitable characteristics to be used in treating infections of the gastrointestinal tract. The granulating step b) has been found to be particularly critical to provide tablets of rifaximin with suitable characteristics.

Indeed, as it is disclosed more in detail in the experimental section, the process of the invention provides tablets that have excellent hardness, disintegration and friability profiles. These physical properties are of paramount importance, as they might have a critical effect on processing and in vivo behavior of the tablet. It has been found that the physical properties obtained thanks to the process of the invention are suitable for industrial scale production and for technological and clinical standpoint. Moreover, as it is disclosed more in detail in the experimental section, the process of the invention provides tablets with proved effectiveness in treating gastrointestinal bacterial infections; accordingly, the pharmacokinetic properties, such as the absorption, of the tablet manufactured by the process of the invention are suitable for being used in treating gastrointestinal bacterial infections.

According to preferred embodiments, the suitable pharmaceutical excipients of step a) can comprise one or more of the following excipients: fillers, disintegrants, binders, and chelating agents. Preferably, the filler is microcrystalline cellulose, the disintegrant is sodium starch glycolate (type A), the binder is hypromellose 2910 (10000 mPa s), and the chelating agent is disodium edetate hydrate. The binders can be added directly during the granulating step b).

Advantageously, other suitable pharmaceutical excipients can be comprised in the mixture and/or added during the process of the invention. For example, one or more glidants such as colloidal silica (anhydrous) can be comprised in the mixture of step a) and/or can be added after the drying step c) to improve the optional mixing of the dry granules before the subsequent step(s) of the process of the invention. Lubricants such as stearic acid can be added before the tableting step d) to improve tableting.

As referred herein and unless otherwise specified, component percentages refer to the weight percentages of the component with respect to the total weight of the uncoated tablet.

It has been found that a mixture comprising the following components is particularly suitable to carry out the process invention:
pseudo-crystalline rifaximin in a range of 40% to 65%, preferably 50% to 60%, more preferably of 55% to 56.3%;
a filler, such as microcrystalline cellulose, in a range of 5% to 40%, preferably of 15% to 30%, more preferably of 20% to 22.5%;
a disintegrant, such as sodium starch glycolate (type A), in a range of 2% to 16%, preferably of 4% to 12%, more preferably of 8% to 8.5%;
a glidant, such as colloidal silica (anhydrous), in a range of 0.5% to 10%, preferably 2% to 8%, more preferably of 4% to 4.2%;
a lubricant, such as stearic acid, in a range of 0.5% to 10%, preferably 1% to 4%, more preferably of 2 to 2.3%;
a binder, such as hypromellose 2910 (10000 mPa s), in a range of 0.5% to 10%, preferably of 1% to 6%, more preferably of 3% to 3.4%; and
a chelating agent, such as disodium edetate hydrate, in a range of 0.5% to 10%, preferably 1% to 4%, more preferably of 2 to 2.8%.

The above-mentioned components may be added in one or more steps of the process of the invention, e.g. based on when they are required. For example, half of the amount of lubricant can be added before the optional compaction c′) discussed below, and the other half of the amount of lubricant can be added before the tableting step d).

The mixture provided in step a) is advantageously in the form of a powder. It can be advantageously mixed before carrying out step b) so that the components are better dispersed in the mixture. The mixture can be mixed directly in the high shear mixer granulator wherein step b) occurs, whereby the granulating step b) can be simply initiated by adding water to the high shear mixer granulator and by setting the relevant parameters of the high shear mixer granulator as disclosed above without unloading the mixture from the mixer.

The wet granulation step b) is preferably carried out using water, more preferably in an amount of 40% to 80%, preferably 60% with respect to the weight of the uncoated tablet.

Drying step c) is preferably carried out until the granules have a loss on drying (LOD) of <8%, more preferably of <6%, even more preferably of <3%. In other words, the endpoint of the drying step c) is when the granules have a LOD value as disclosed above. It has been found that these values provide an effective tableting step, whereby the tablets of rifaximin obtained possess suitable physical and pharmacokinetic properties.

The drying step (step c)) is preferably carried out at a temperature comprised in the range of 50 to 100° C., more preferably of 60 to 90° C., even more preferably of 70 to 80° C., even more preferably is 75° C.

Drying of the granules (step c)) can be carried out by any means know in the art. Preferably, the granules can be dried with the aid of flow of air, more preferably a heated flow of air at the temperatures above disclosed. In particularly preferred embodiments, drying of granules (step c)) is carried out in a fluid bed, wherein the temperature of the inlet air is the temperature as above disclosed.

The dry granules can be advantageously sieved after step c), preferably through a screen of at least 6 mm, more preferably of at least 4 mm, even more preferably through a 2 mm screen. This improves the homogeneity of the granules and improves the subsequent steps as well. After sieving, it can be advantageous to weight the granules and calculating the yield thereof so that further components that can be added later, such as lubricants, glidants and/or the components of a coating composition, are added according to the weight of the remaining granules, i.e. the sieved granules.

A further step c′) can be advantageously be comprised in the process of the invention, such further step c′) being carried out after step c) (and after sieving, if carried out) and before step d). The further step c′) is a compaction step, wherein the dry granules are compacted into slugs. After the compaction step c′), the obtained slugs are compressed into the tablets of rifaximin according to step d) of the process of the invention. Lubricants such as stearic acid can be advantageously added before step c′) to improve the compaction step. Glidants such as colloidal silica (anhydrous) can also be added, in particular if the dry granules have to be mixed before step c′) to improve dispersion of the optionally added lubricants.

If the further step c′) is carried out, sieving of the obtained slugs can be advantageously carried out before step d). In particular, sieving the slugs through a screen of at least 5 mm, more preferably of at least 3 mm, even more preferably through a 1.6 mm screen, can result in high homogeneity slugs, thereby improving the subsequent steps. After sieving, it can be advantageous to weight the slugs and calculating the yield thereof so that further components that can be added later, such the components of a coating composition, are added according to the weight of the remaining slugs, i.e. the sieved slugs.

The tableting step d) can be carried out with conventional tablet presses.

An advantageous parameter of the tableting step d) is a compression force of 10 to 30 KN, preferably of 15 to 20 KN.

After the tableting step d), and therefore after obtaining the tablet of rifaximin, it can be advantageous to coat the obtained tablets with a further step d′) (i.e. a coating step), whereby coated tablets are obtained. Accordingly, further excipients are added to obtain the coated tablets; in particular, one or more of the following excipients can be added: coating polymers, plasticizers, anti-adherents, colorants and opacifier. A preferred coating composition comprises the following components in the following amount:
a coating polymer, such as hydroxypropylmethylcellulose (HPMC), in a range of 0.5% to 10%, preferably of 1% to 6%, more preferably 3% to 3.4%;
a plasticizer, such as polyethylene glycol, in a range of 0.05% to 1%, preferably of 0.1% to 0.6%, more preferably 0.3%;
an opacifier, such as titanium oxide, in a range of 0.05% to 1%, preferably of 0.1% to 0.4%, more preferably 0.2%;
an anti-adherent, such as talc, in a range of 0.05% to 1%, preferably of 0.1% to 0.6%, more preferably 0.3%; and
q.s. of iron oxide.

Water, aqueous solutions and other suitable solvents for pharmaceutical use can be used as solvents for coating. The coating step d′) can be carried out in any conventional coating apparatus, for example a pan coater and fluid bed equipment. The optional step of coating of the present invention (step d′)) improves the mechanical resistance of the tablets, the compliance of the patients and masks the taste of rifaximin, thereby improving the overall characteristics of the tablet.

Further subject-matter of the present invention is a tablet of rifaximin obtainable with the process of the invention.

As stated above, the tablet of the invention is obtainable starting from pseudo-crystalline rifaximin, which has many advantages over known rifaximin crystalline forms.

As it can be observed in the experimental section, the tablet of the invention has been found to be particularly useful as an antibiotic for the gastrointestinal tract. Indeed, the tablet of the invention has been found bioequivalent to a rifaximin medicinal marketed in Europe with indications for treating and preventing bacterial infections in the gastrointestinal tract.

Some known crystalline forms of rifaximin cannot be formulated into an oral dosage form as they tend to be absorbed too much after oral administration to exert the antibiotic effect inside the gastrointestinal tract. On the contrary, the pseudo-crystalline rifaximin, when formulated with the process of the invention, has been found to be suitable to provide a tablet of rifaximin, i.e. the tablet of the invention, which has suitable pharmacokinetic properties (as well as mechanical properties) so that the rifaximin can exert its antibiotic properties inside the gastrointestinal tract.

The tablet of the invention comprises rifaximin as the active principle ingredient and one or more of the following excipients: fillers, disintegrants, binders, lubricant, glidants, and chelating agents.

According to embodiments, the tablet of the invention can be coated, for example according the coating step d′) disclosed above. When the tablet of the invention is coated, the tablet can further comprise, in particular in its coating, one or more of the following excipients: coating polymers, plasticizers, colorants, anti-adherents, and opacifiers.

According to embodiments, the tablet of the invention has a hardness comprised in the range of 70 N to 200 N, preferably of 100 N to 150 N; hardness may depend on the amount of pseudo-crystalline rifaximin comprised in the composition. Hardness is measured with SOTAX HT 100 instrument according to Pharmacopoeia specifications.

According to embodiments, the tablet of the invention has a disintegration of less than 20 min, preferably of less than 10 min. Disintegration is measured with SOTAX DT2 instrument according to Pharmacopoeia specifications.

According to embodiments, the tablet of the invention has a friability of less than 1%, preferably of less than 0.1%. Friability is measured with SOTAX F2 instrument according to Pharmacopoeia specifications.

### DESCRIPTION OF THE FIGURES

FIG. 1 illustrates the XRPD pattern of the pseudo-crystalline rifaximin used as a starting material for the process and the tablet of the invention. This XRPD pattern was obtained using a SIEMENS D5000 instrument and under the following working conditions:

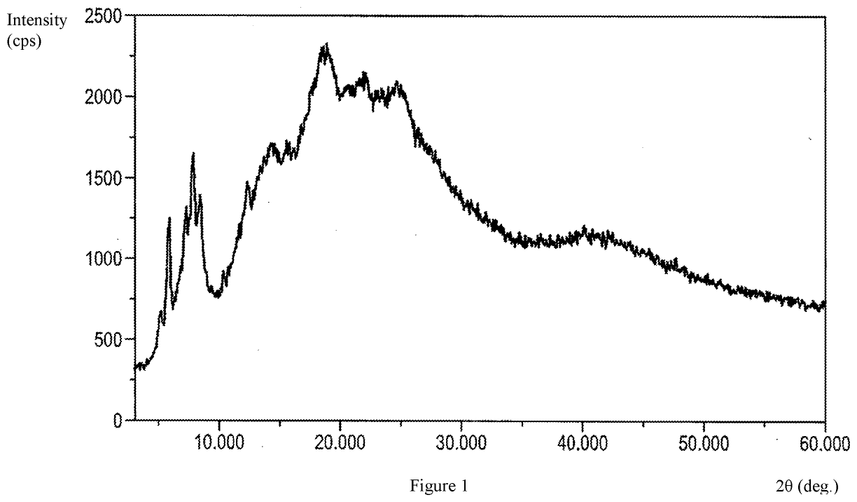

radiation used: Kα of Copper (λ=1.5406 Angstrom);

tension of the generator: KV 35;

current of the generator: 30 mA; and

starting and final angular 20 value: from 2.0° to 60.0°.

FIG. 2 illustrates the complete list of peaks of the XRPD pattern obtained as above disclosed of the pseudo-crystalline rifaximin.

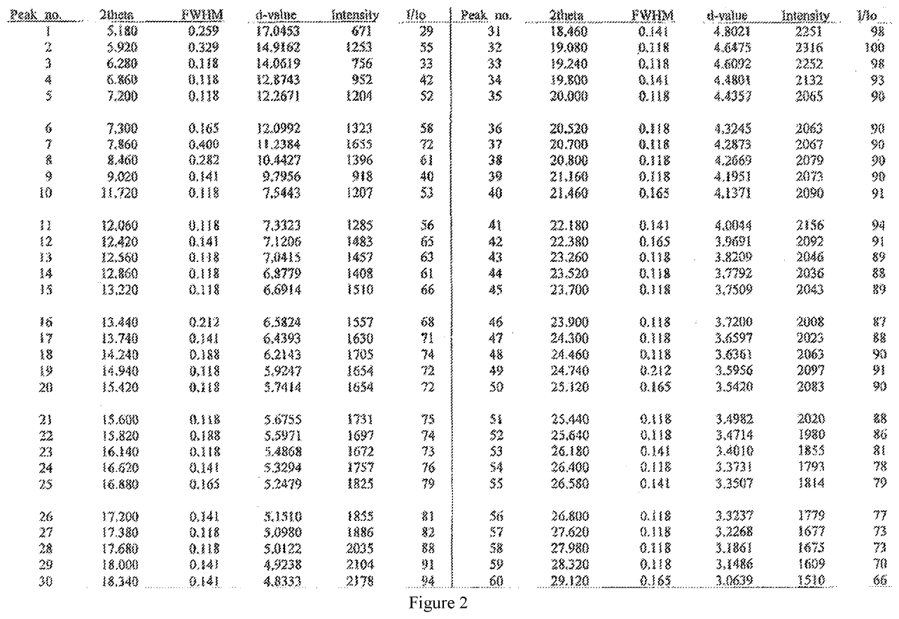

The present invention is further disclosed in detail by means of the following examples, which are meant for illustrative purpose only or for demonstrating the effectiveness of the subject-matter of the invention. Accordingly, the following examples do not aim to limit the scope of the invention.

### EXPERIMENTAL SECTION

### Example 1—Manufacture of a Tablet of Rifaximin (200 mg) According to the Process of the Invention

In the present example, a coated tablet containing 200 mg of rifaximin is manufactured.

A mixture comprising 200.00 mg of rifaximin (56.3%), 80.00 mg of cellulose microcrystalline (22.5%), 30.00 mg of sodium starch glycolate (type A) (8.5%), 12.00 mg of hypromellose 2910 (HPMC) (10000 mPa s) (3.4%), and 10.00 mg of disodium edetate hydrate (2.8%), is loaded into a high shear mixer granulator (GEA) and is mixed for 5 minutes by setting the impeller at 600 rpm and the chopper at 3000 rpm.

The granulation step is started by adding 213 mg (60.0%) of purified water to the high shear mixer granulator and by setting the impeller at 1500 rpm and the chopper at 3000 rpm. The granulation is ended after 15 minutes.

The granules are transferred to a fluid bed (VANGUARD) wherein drying occurs. The temperature of the inlet of the fluid bed is set at 75° C. and the temperature product reaches 45° C. The drying is ended when the LOD is <3%.

After drying, the dry granules are sieved through a 2 mm screen.

The dry granules are transferred to a mixer (SERVOLIFT BIN BLENDER), and 15.00 mg of colloidal silica (anhydrous) (4.2%) and 4 mg of stearic acid (1.15%) are added to the mixer. The mixer is set to 6 rpm and mixing is ended after 20 minutes.

The granules are transferred to a tablet press for the compaction step (KORSCH EK0 TABLET PRESS). Slugs are obtained.

Slugs are sieved through a 1.6 mm screen.

Slugs are transferred to a mixer (SERVOLIFT BIN BLENDER), and 4 mg of stearic acid (1.15%) is added to the mixer. The mixer is set to 6 rpm and mixing is ended after 20 minutes.

The slugs are transferred to a tablet press (KORSCH PH 106 TABLET PRESS). The pressure applied to carry out the tableting step is 15-20 KN. Tablets of rifaximin of the invention are obtained.

The tablets of rifaximin are transferred to a pan coater and the following excipients are added: 12.00 mg of hypromellose 2910 (HPMC) (6 mPa s) (3.4%), 1.20 mg of polyethylene glycol 6000 (0.3%), 0.80 mg of titanium oxide (0.2%), 1.00 mg of talc (0.3%), and q.s. of iron oxide. Coating is started by adding 100.00 mg of purified water (28.2%) and by setting the inlet air at 50° C. The coating is ended after 70 min.

The tablets produced according to Example 1 have the following characteristics measured as disclosed above:
hardness: 100 N;
disintegration: <10 min;
friability: <0.1%.

According to the above, the mechanical properties of the tablets produced by the process of the invention are suitable for marketing and administration to patients.

### Example 2—Manufacture of a Tablet of Rifaximin (550 mg) According to the Process of the Invention

In the present example, a coated tablet containing 550 mg of rifaximin is manufactured.

The tablet comprising 550 mg of rifaximin is obtained with same procedure of Example 1, but with the different amounts of components indicated below.

The starting mixture comprises: 550.00 mg of rifaximin (56.3%), 220.00 mg of cellulose microcrystalline (22.5%), 82.50 mg of sodium starch glycolate (type A) (8.5%), 33.00 mg of hypromellose 2910 (HPMC) (10000 mPa s) (3.4%), and 27.50 mg of disodium edetate hydrate (2.8%); 585.75 mg of purified water (60%) is added for the granulation step.

The following excipients are added before the compaction step: 41.25 mg of colloidal silica (anhydrous) (4.2%) and 11.00 mg of stearic acid (1.15%). The following excipient is added before the tableting step: 11.00 mg of stearic acid (1.15%).

The following excipients are added for the coating step: 33.00 mg of hypromellose 2910 (HPMC) (6 mPa s) (3.4%), 3.30 mg of polyethylene glycol 6000 (0.3%), 2.20 mg of titanium oxide (0.2%), 2.75 mg of talc (0.3%), q.s. of iron oxide, and 275.00 mg of purified water (28.2%).

The tablets produced according to Example 2 have the following characteristics measured as disclosed above:
hardness: 150 N;
disintegration: <10 min;
friability: <0.1%.

According to the above, the mechanical properties of the tablets produced by the process of the invention are suitable for marketing and administration to patients.

### Example 3—Bioequivalence Test

A bioequivalence test comparing the tablet of the invention comprising 200 mg of rifaximin with a tablet of rifaximin currently marketed in Europe, namely NORMIX® tablet (200 mg) marketed by ALFASIGMA S.P.A. (Italy), is carried out. NORMIX® 200 mg tablet is indicated for acute and chronic intestinal infections by gram-positive and gram-negative bacteria; diarrheal syndromes; diarrhea from altered balance of intestinal microbial flora (traveler's diarrhea, enterocolitis); pre- and postoperative prophylaxis of infectious complications in surgery of the gastrointestinal tract; and adjuvant in the treatment of hyperammonemia.

The test is designed as a randomized trial. It is carried out on a group of 52 healthy subjects divided in two groups A and B. In the first part of the present test, NORMIX® has been administered to the subjects of group A and the tablet of the invention has been administered to the subjects of group B. In the second part of the test, the tablet of the invention has been administered to the subjects of group A and NORMIX® has been administered to the subjects of group B. The washout period between first part and second part is 1 week. Sampling time is 0, 0.25, 0.5, 0.75, 1, 1.25, 1.5, 1.75, 2, 3, 4, 6, 8, 12 and 24 hours after administration. The blood samples are analyzed with LC-MS/MS.

The results of the present test are showed on Table 1:

TABLE 1

| / | AUCₜ (pg hr/ml) | Cₘₐₓ (pg/ml) |
|---|---|---|
| Tablet of the invention | 5040.46 ± 1002.58 | 991.56 ± 351.15 |
| (200 mg)* | | |
| NORMIX ®* | 5260.72 ± 1194.20 | 1012.57 ± 350.31 |
| 90% confidence interval | 0.9176 ≤ δ ≤ 1.0164 | 0.8979 ≤ δ ≤ 1.0674 |
| (δ)** | | |

*Mean ± SD
**Log conversion

Dissolution tests and bioavailability studies comparing the tablet of the invention and NORMIX® provided equal (comparable) results.

According to the above results, the tablet of the invention, manufactured with the process of the invention, has resulted to be bioequivalent to NORMIX®, which is a tablet of rifaximin with a proven history to be effective in treating many pathologies, such as gastrointestinal bacterial infections. Accordingly, the tablet of rifaximin of the invention has been proved to be effective in treating gastrointestinal bacterial infections and to be suitable to treat every medical condition to which NORMIX® is indicated.

1. A process for the manufacturing of a tablet of rifaximin comprising the following steps:
a) providing a mixture comprising pseudo-crystalline rifaximin and suitable pharmaceutical excipients;
b) granulating the mixture of step a) by wet granulation in a high shear mixer granulator, whereby granules are obtained;
c) drying the granules obtained in step b), whereby dry granules are obtained; and
d) tableting the dry granules of step c), whereby said tablet of rifaximin is obtained;
wherein the granulating step b) is carried out with an impeller speed of 1000 rpm or more and for a time longer than 10 minutes.

2. The process according to claim 1, wherein the impeller speed is comprised in the range of 1000 to 2000 rpm.

3. The process according to claim 1, wherein step b) is carried out for a time comprised in the range of 10 to 20 minutes.

4. The process according to claim 1, wherein said suitable pharmaceutical excipients of step a) are one or more of the excipients selected from the group of: fillers, disintegrants, binders, glidants, lubricants and chelating agents.

5. The process according to claim 1, wherein the mixture comprises:
pseudo-crystalline rifaximin in a range of 40% to 65%;
a filler, such as microcrystalline cellulose, in a range of 5% to 40%;
a disintegrant, such as sodium starch glycolate (type A), in a range of 2% to 16%;
a glidant, such as colloidal silica (anhydrous), in a range of 0.5% to 10%;
a lubricant, such as stearic acid, in a range of 0.5% to 10%;
a binder, such as hypromellose 2910 (10000 mPa s), in a range of 0.5% to 10%;
a chelating agent, such as disodium edetate hydrate, in a range of 0.5% to 10%.

6. The process according to claim 1, wherein the drying step c) is carried out until the granules have a loss on drying (LOD) of <8%.

7. The process according to claim 1, wherein the drying step c) is carried out at a temperature comprised in the range of 50 to 100° C.

8. The process according to claim 1, wherein a further compaction step c′) is carried out after step c), said compaction step c′) comprising compacting the dry granules into slug.

9. The process according to claim 1, wherein a further coating step d′) is carried out after the tableting step d), said coating step d′) comprising coating said tablet of rifaximin obtained in step d).

10. The process according to claim 9, wherein the coating step d′) is carried out using the following coating composition:
a coating polymer in a range of 0.5% to 10%;
a plasticizer in a range of 0.05% to 1%;
an opacifier in a range of 0.05% to 1%;
an anti-adherent in a range of 0.05% to 1%; and
q.s. of iron oxide.

11. A tablet of rifaximin obtainable with the process according to claim 1.

12. The tablet according to claim 11, having a hardness comprised in the range of 70 N to 200 N; a disintegration of less than 20 min; and a friability of less than 1%.

13. The process according to claim 1, wherein the impeller speed is 1500 rpm.

14. The process according to claim 1, wherein step b) is carried out for a time of 15 minutes
